(19) United States
(12) Patent Application Publication
(10) Pub. No.: US 2016/0226542 A1
(43) Pub. Date: Aug. 4, 2016

(54) WEARABLE SYSTEM

(71) Applicants: Bao Tran, Saratoga, CA (US); Ha Tran, Saratoga, CA (US)

(72) Inventors: Bao Tran, Saratoga, CA (US); Ha Tran, Saratoga, CA (US)

(21) Appl. No.: 15/096,041

(22) Filed: Apr. 11, 2016

Related U.S. Application Data
(63) Continuation of application No. 14/471,489, filed on Aug. 28, 2014.

(51) Int. Cl. H04B 1/3827; A61B 5/00; A61B 5/11; A61B 5/053; A61B 5/0404; H04W 4/00; A61B 5/01; A61B 5/091; A61B 5/145; A61B 5/0488; A61B 5/0476; A61B 5/024; A61B 5/021

(52) U.S. Cl. CPC H04B 1/385; A61B 5/02438; A61B 5/681; A61B 5/1118; A61B 5/053; A61B 5/0404; A61B 5/02141; A61B 5/01; A61B 5/091; A61B 5/14542; A61B 5/4872; A61B 5/0488; A61B 5/0476; H04W 4/005

(57) ABSTRACT

A wearable device includes a band having a flexible battery integrally formed therein; a processor powered by the flexible battery; a cellular transceiver to make a phone call; a WiFi transceiver and personal area network (PAN) transceiver coupled to the processor; a microphone to receive user speech and a speech recognizer to process user speech or provide voice control; and a health application communicating health application data with a remote computer, wherein the health application includes computer readable code to store and analyze user fitness information including one or more of: posture, ambulation, location position, sleep, exercise, calorie, or medicine compliance.

### BACKGROUND

The present invention relates to a wearable device.

While digital watches are inexpensive and provide great utility/convenience, many people still prefer high end watches such as the Philipe Patek or Rolex watches to wear as jewelry or to show their status. On a parallel note, for users looking to lose weight, increase physical activity, or simply improve overall health, a personal activity tracker can help reach their goal. Seeing how much activity is done (or not) get, day-by-day and week-by-week, could motivate the user to start taking the stairs or walking the dog an extra lap around the block. At the very least, it will make the user more mindful of his or her activity level, which is a huge first step to getting fit. Smart wearable devices on the market today are highly evolved cousins of pedometers from yesteryear. They're much smarter, more accurate, and do a whole lot more than measure how much the user walks. Paired with a companion Web account, mobile app, and maybe a few auxiliary devices, wearable devices give better insight than analog watches into the habits that make up the user's lifestyle, including sleep, calorie consumption, heart rate, blood pressure, and more.

### SUMMARY

In one aspect, a wearable device includes a band having a flexible battery integrally formed therein; a processor powered by the flexible battery; a cellular transceiver to make a phone call; a WiFi transceiver and personal area network (PAN) transceiver coupled to the processor; a microphone to receive user speech and a speech recognizer to process user speech or provide voice control; and a health application communicating health application data with a remote computer, wherein the health application includes computer readable code to store and analyze user fitness information including one or more of: posture, ambulation, location position, sleep, exercise, calorie, or medicine compliance.

In another aspect, a wrist-worn device includes an analog watch and a digital wearable device connected by wrist band(s). In implementations, the wrist band can generate or store power for the digital device. The digital wearable device includes a heart sensor or a heart rate sensor. The digital wearable device can include a pedometer, a positioning system, or an exercise tracker. A rotor can generate electricity during movement. The analog watch comprises an energy harvesting device to generate electricity from movement or from solar energy. The analog watch is adjusted through the digital watch with a motor, wherein the analog watch changes time during a time-zone change. The digital watch changes time during a time-zone change. One or more wrist bands coupling the analog watch and the digital device. The wrist bands store or generate energy for the digital wearable device. The digital wearable device communicates with a telephone over a personal area network. The digital device displays emails, texts and alerts on a touch sensitive screen to dismiss calls and alarms with a tap. The device can have a curved display contouring with a wearer wrist, and secured to the analog watch with interchangeable bands and multiple color options. The wrist bands store or generate electrical energy. A magnetic pendulum rotor rotatably coupled to a shaft above a planar metal.

Advantages of the above device may include one or more of the following. Professionals get the pleasure of having a high end jewelry on their wrist, yet the convenience and information power of a digital wearable device. Such digital device can relay information such as caller information, social network messages from Facebook for example, calendar reminders. The system can interact with cellular phones to receive notifications such as texts and email messages. The user can then open that message from the watch to save the user the hassle of looking through the apps on the phone to find it. The device can link to a cellular phone and when the phone is lost, the watch has the ability to connect to the phone and cause vibrations to assist in finding the phone. The phone can also help find the system with the same method. When the user's phone leaves the area of the system, it locks the phone automatically and will unlock when the phone comes back into range of the watch. Calls can be made from the wearable device so that the user doesn't need to use a phone.

### BRIEF DESCRIPTION OF THE DRAWINGS

FIG. 1A shows a top view of a device with two watches to be mounted on both sides of a wrist.

FIGS. 1B and 1C show a side view of the watch of the device of FIG. 1A.

FIG. 1D shows a bottom view of the device of FIG. 1A.

FIG. 2 shows an attachment mechanism to secure the analog watch one to the wristband 112.

FIGS. 3A-3B show an exemplary energy harvesting system.

FIG. 4A shows a perspective view of a wearable watch with a wrist band having a flexible battery unit integrally formed therein, according to embodiments described herein;

FIG. 4B shows another perspective view of a wearable watch with a wrist band having a flexible battery unit integrally formed therein, according to embodiments described herein;

FIG. 4C shows a perspective view of the flexible battery unit, according to embodiments described herein.

FIG. 4D is a diagram illustrating generally, communication between wearable watch and smart phone, according to embodiments described herein.

FIG. 5 is a diagram illustrating generally, communication between the wearable watch and implantable device, according to embodiments described herein.

FIG. 6 shows a graphical overview of a wearable watch system 600, according to embodiments described herein.

### DESCRIPTION

The present invention offers the advantages of an analog watch with a digital wearable device that, together with a smart phone, augments the analog watch with instant notifications of emails, SMS, incoming calls, and 3rd party apps to keep the wear well connected. The entire system provides the elegance of high end watches that serve as status symbol and jewelry, while providing smart watch capability with personalized real-time information on the progress and results of the workout by deploying the device's optical heart rate sensor. Personalized and adaptive next workout recommendation will help the user to exercise according to the user's individual needs.

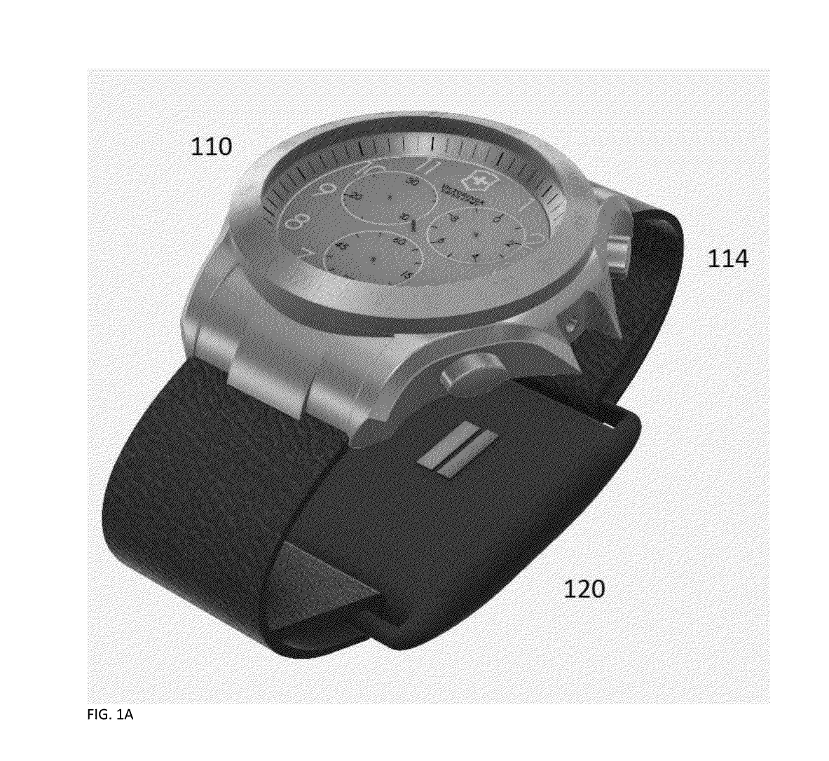

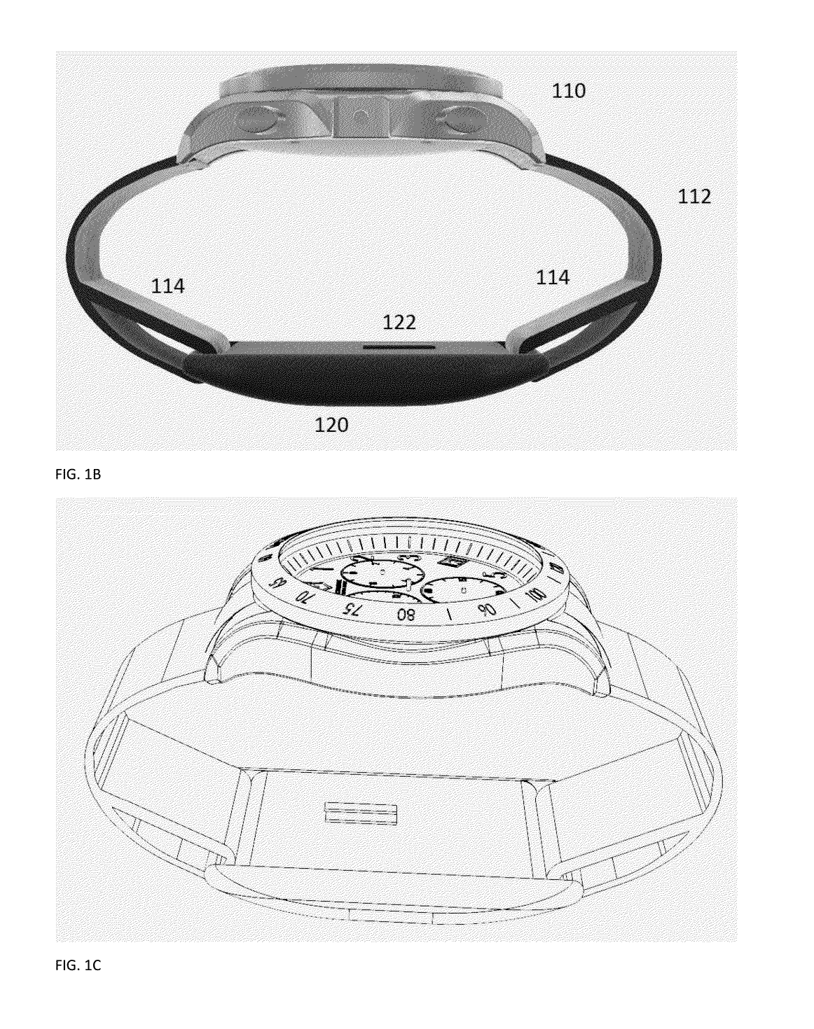

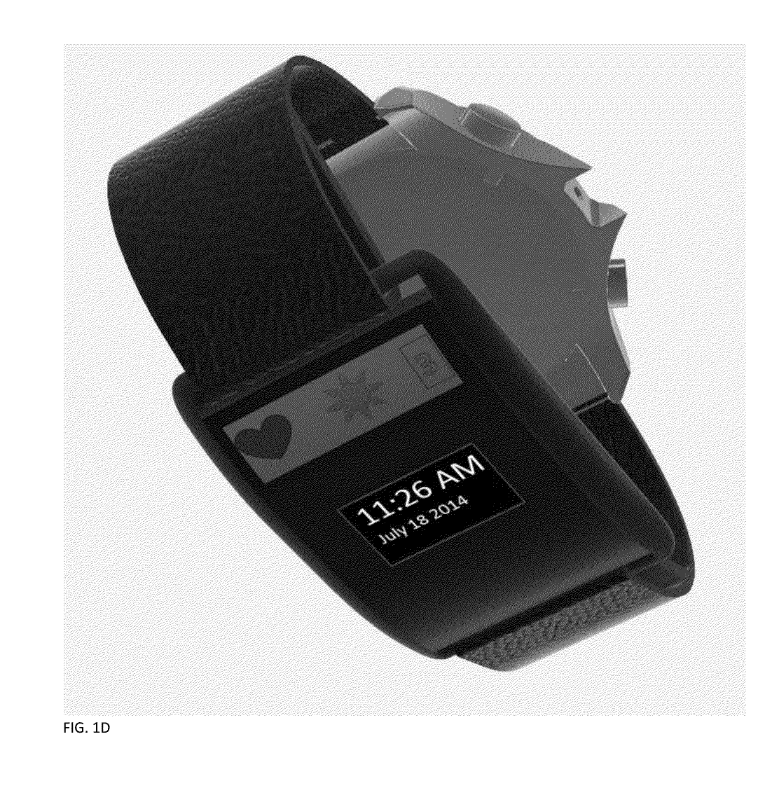

FIGS. 1A-1D show a hybrid watch system with an analog watch supported by a digital wearable device that can be worn on the back of the wrist or as the strap for a conventional analog watch 110. The analog watch 110 has position oscillation mechanisms such as those found in high-end devices that many people use as jewelry. One embodiment uses an eccentric weight, called a winding rotor, which rotates with the movement of the wearer's wrist. The back-and-forth motion of the winding rotor couples to a ratchet to wind the mainspring automatically. Self-winding watches usually can also be wound manually to keep them running when not worn or if the wearer's wrist motions are inadequate to keep the watch wound. Another embodiment uses electronic movements, also known as quartz movements, which has few or no moving parts, except a quartz crystal which is made to vibrate by the piezoelectric effect. A varying electric voltage is applied to the crystal, which responds by changing its shape so, in combination with some electronic components, it functions as an oscillator. It resonates at a specific highly stable frequency, which is used to accurately pace a timekeeping mechanism. Most quartz movements are primarily electronic but are geared to drive mechanical hands on the face of the watch in order to provide a traditional analog display of the time, a feature most consumers still prefer.

On the other side of the watch or embedded into the watch strap is a digital wearable device 120. In one embodiment the digital device 120 contains a heart rate sensor 122 that faces the user's wrist to sense blood flow with the wrist. A band 112 links the digital device 120 securely to the analog watch 110. The band 112 goes through one end of the digital device 120 and forms a loop 114 that is attached to the band 112.

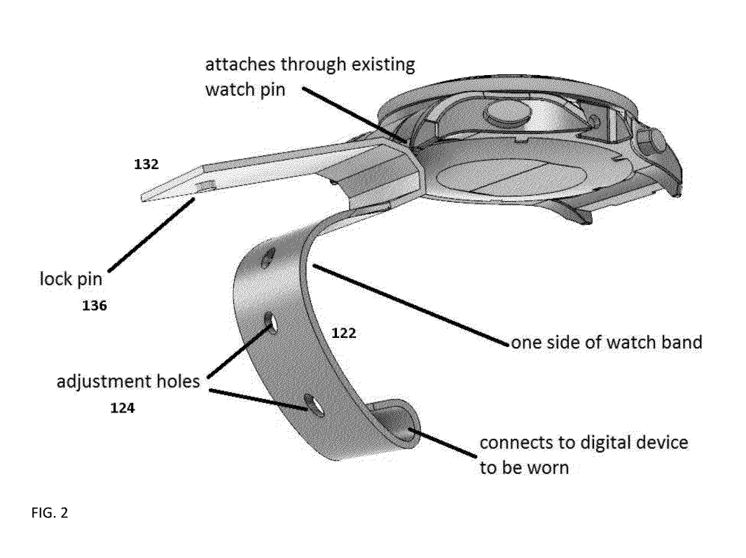

FIG. 2 shows an exemplary attachment method to fasten the watches to the wrist with metal bands. In this embodiment, a metal band 122 with a plurality of adjustment holes 124 is used. One end of the metal band 132 is shaped to hook to the device device, while the other end 134 is a multi-segmented metal portion that can flexibly be looped through the analog watch wrist strap and the end 134 includes a lock pin 136 that can be pushed into one of the adjustment holes 124.

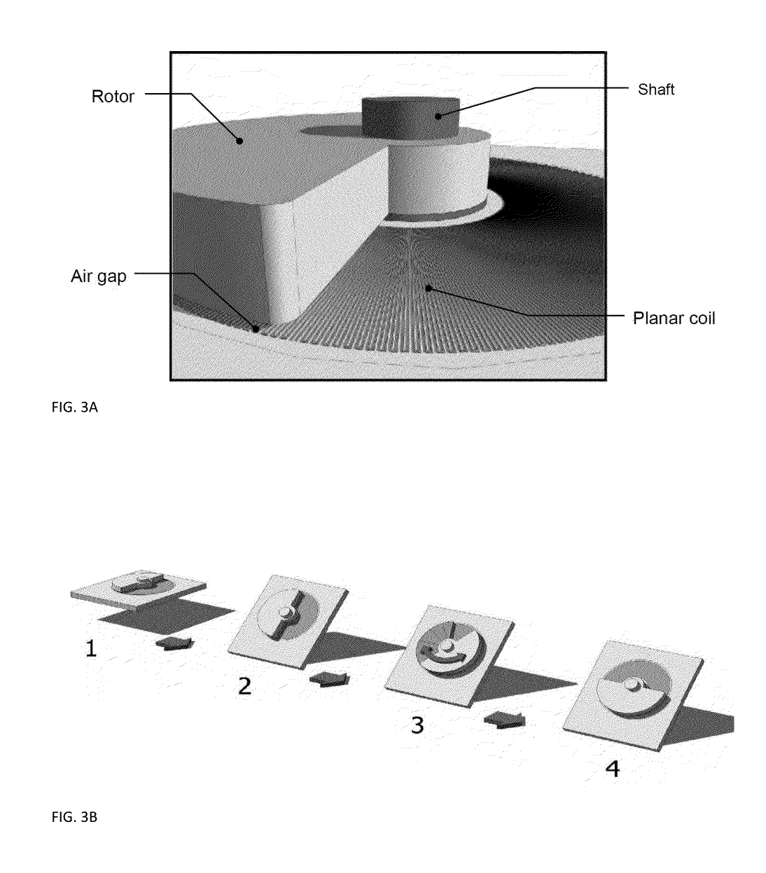

FIG. 3A-3B show an exemplary energy harvesting system with a rotor. The system of FIG. 3A has a magnetic pendulum rotor rotatably coupled to a shaft above a planar metal. When the asymmetrical pendulum leaves its initial stable position due to the motion of some body organ, it oscillates for a certain time to finally reach a new stable position. In the process, a changing axial magnetic field cutting through the underneath planar copper coil induces a voltage across its terminals to generate energy. In FIG. 3B, the initial stable state 1 is excited by movement as state 2, driving the rotation in state 3 to a new stable state 4.

Another embodiment herein discloses a watch adapted to be worn on a user's wrist. The watch includes a dial including a control circuit. A flexible battery unit, being disposed on wrist band and connecting to the control circuit electrically. Unlike conventional systems, the flexible battery is integrated into the wrist band, such as to be worn by the user. The flexible battery unit can be configured to provide an output power to the watch through the control circuit. Further, the watch can be configured to access a wide area network through a smart phone, such as to provide information about weather, notifications, schedule, time, and the like. The wrist can be a natural place for wearable technology in the form of watches. The wearable watch described herein can be used in various applications in numerous spaces, such as medical devices, personal fitness monitors, smart badges that automatically exchange contact information, and measure social interaction, and the like.

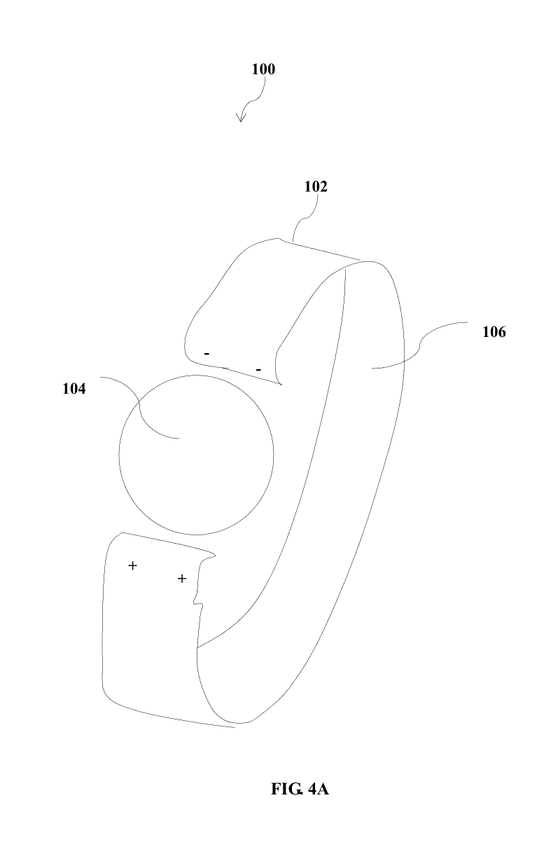

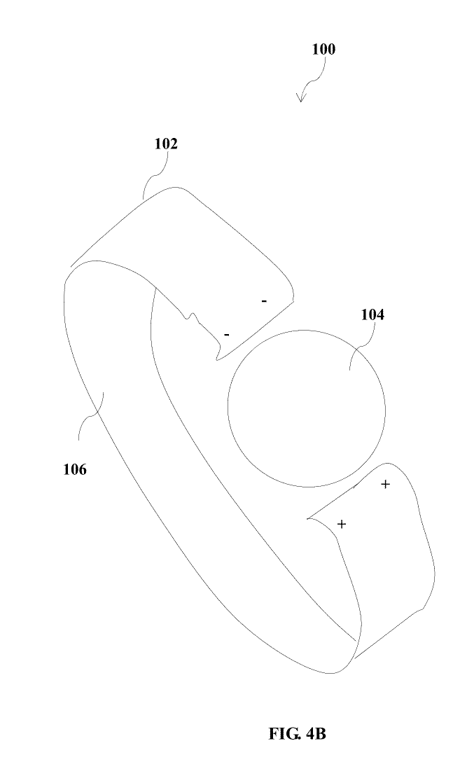

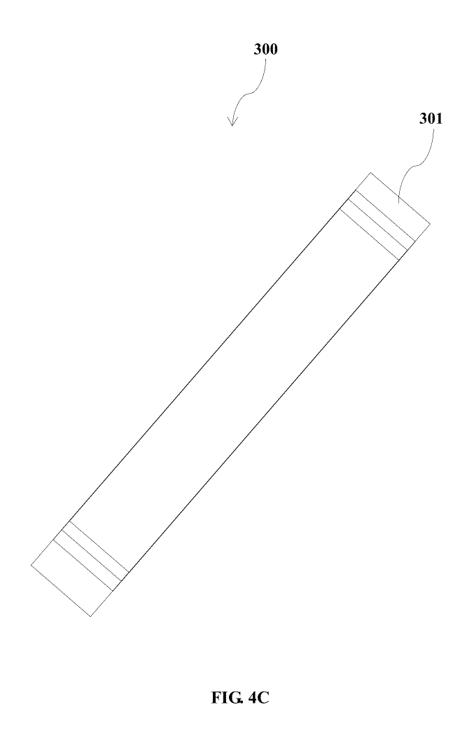

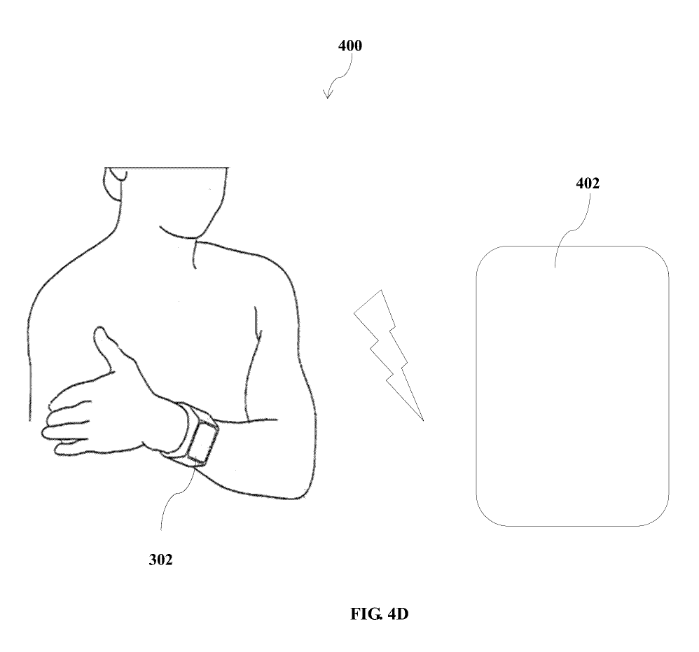

FIG. 4A shows a perspective view of a wearable watch 100 with a wrist band 102 having a flexible battery unit integrally formed therein, according to embodiments described herein. In an embodiment, the wearable watch 100 can be configured to include electronic unit intended to be worn, for example, on a user's wrist. The watch 100 may be adapted for personal communication or personal computing. In an embodiment, the watch 100 can be configured to include a watch face 104 as well as, for example, speaker, microphone, and the like components.

One embodiment of the watch uses flexible substrates to embed display and processing electronics including multicores printed thereon. One implementation uses on roll-to-roll systems like those used to print newspapers, potentially enabling cost-cutting volume production. The flexible display can provide inductive screen that uses magnetized styluses to induce a field in a sensing layer at the back of the display. The display uses as a backplane material a thin-film plastic material made by DuPont called Teonex polyethylene napthalate (PEN) which provides support for the display while allowing the inductive touch layer to work.

In one embodiment, a substrate is provided and an electronic ink is printed onto a first area of the substrate. The present invention takes advantage of the physical properties of an electronic ink which permits a wide range of printing and coating techniques to be used in creating a display. An electronic ink is an optoelectronically active material which comprises at least two phases: an electrophoretic contrast media phase and a coating/binding phase. The electrophoretic phase comprises, in some embodiments, a single species of electrophoretic particles dispersed in a clear or dyed medium, or more than one species of electrophoretic particles having distinct physical and electrical characteristics dispersed in a clear or dyed medium. The coating/binding phase includes, in one embodiment, a polymer matrix that surrounds the electrophoretic phase. In this embodiment, the polymer in the polymeric binder is capable of being dried, crosslinked, or otherwise cured as in traditional inks, and therefore a printing process can be used to deposit the electronic ink onto a substrate. An electronic ink is capable of being printed by several different processes, depending on the mechanical properties of the specific ink employed.

The optical quality of an electronic ink is quite distinct from other electronic display materials. The most notable difference is that the electronic ink provides a high degree of both reflectance and contrast because it is pigment based (as are ordinary printing inks). The light scattered from the electronic ink comes from a very thin layer close to the top of the viewing surface. In this respect it resembles a common, printed image. Thus, electronic ink is easily viewed from a wide range of viewing angles in the same manner as a printed page. Such ink approximates a Lambertian contrast curve more closely than any other electronic display material. Since electronic ink can be printed, it can be included on the same surface with any other printed material. Electronic ink can be made optically stable in all optical states, that is, the ink can be set to a persistent optical state. Fabrication of a display by printing an electronic ink is particularly useful in low power applications because of this stability.

Graphene devices can be formed on the flexible display to improve processing speed. One implementation uses chemical vapor deposition to deposit a 300-nanometer-thick layer of nickel on top of a silicon substrate. Next, the process heats this substrate to 1,000 C.° in the presence of methane, and then cool it quickly down to room temperature. This leaves behind graphene films containing six to ten graphene layers on top of the nickel. By patterning the nickel layer, the system create patterned graphene films. The films can be transferred to flexible substrates while maintaining high quality. The transfer is done in one of two ways. One is to etch away the nickel in a solution so that the graphene film floats on its surface, ready to be deposited on any substrate. A simpler trick is to use a rubber stamp to transfer the film.

Glass can be used as a surface for building thin-film devices on. Glass is impermeable and its surface is also very smooth, which means it's much easier to build perfectly structured, high-performance electronics on top of it.

Technologies such as printable electronics (EP), electronic paper (or ePaper), other flexible and organic-based electronics, other thin film electronics, combinations thereof, and the like, are particularly well-suited for all or part of the electronic circuitry. The stretchable aspect of printable electronics makes printable electronics a well-suited choice for the electronic circuitry in wearable applications. Additionally, the flexible, sunlight viewable and low cost aspects of ePaper make ePaper a well-suited choice for disposable bandage applications. As a result, electronic components such as resistors, transistors, diodes, memory cells, and the like are capable of being formed (e.g., via jetted ink in a reliable, consistent, low cost process) on a flexible paper-style medium for integration and electrical operation within the wearable electronic.

In some arrangements, the flexible electronic controller further includes display circuitry which is constructed and arranged to provide visual output. Again, technologies such as printable electronics, ePaper, and the like are well-suited for such circuitry. In some arrangements, the display circuitry provides a chameleon function by matching the underlying skin color for aesthetic and/or camouflage purposes. In some arrangements, the display circuitry selectively transitions between being opaque and transparent (e.g., in response to a switch or button press of a micro-actuator, touch or stretch programmed, etc.) to enable a user to view a surface under the wearable device.

In some arrangements, the flexible electronic controller 34 further includes material storage 108. In some arrangements, such storage source 108 includes multiple chemicals such as medicines, adhesive, anti-adhesive, etc. In some arrangements, the storage 108 is capable of collecting tissue material (e.g., chemicals from the skin for the purpose of later mixing and concentrating with other chemicals, chemical/bacterial/viral collection for chemical analysis following bandage removal, etc.).

In some arrangements, the flexible electronic controller further includes a memory which is constructed and arranged to store data. In some arrangements, the memory is implemented using printable electronics, or the like and stores digital/binary information. The memory can be volatile storage (e.g., requiring periodic refreshing) or non-volatile storage (e.g., magnetically or flash based).

In one embodiment, a single transfer process is employed in which thin flexible integrated circuit elements are fabricated within a single crystal silicon layer formed over an insulated silicon substrate (SOI). The circuit is overlaid with an encapsulating layer before being transferred onto a second substrate such as glass. The second substrate is provided with a layer of adhesive on the contact surface and a separation layer or etch stop, such as a copper film between the substrate and adhesive layer. The integrated circuit is then transferred onto the second substrate such that the encapsulating layer bonds with the adhesive layer. The silicon substrate is then removed to expose a silicon oxide layer that can be used as an insulating layer on a silicon wafer. Portions of the silicon oxide layer are then removed to expose the contacts of the integrated circuits or to further process the integrated circuit. Similarly, the second substrate is released at the separation layer to yield a circuit structure having thickness in the range of 0.1 to 100 microns or more, depending upon the specific application. For most applications, a transferred circuit structure of less than 20 microns is preferred. For many CMOS circuit applications silicon films preferably have a thickness in the range of 0.3-1.5 microns. In the final structure, the separation layer, such as copper, can remain within the structure to provide support, electrical shielding, thermal control and/or grounding, or alternatively, can be removed. Upon removal of the second substrate the circuit is supported with the adhesive layer which bends readily. The device is then completed, sealed and any necessary external connectors or bonding pads are completed or exposed, respectively. The adhesive can be commercially available epoxies such as Tracon or EP-112, or a thermally conductive epoxy such as EP-30AN having aluminum nitride particles suspended therein. The adhesive layers employed herein can have varying thickness up to 75 microns or more.

In another embodiment, a double transfer process is employed in which the circuits are transferred to an intermediate substrate before being transferred to a third substrate. In the preferred embodiment, flexible integrated circuit devices are fabricated and then covered with an encapsulating layer. The structure containing the integrated circuits is then transferred onto a second substrate so that the encapsulating layer bonds with a layer of adhesive. A thin layer of amorphous silicon serving as a release layer and/or etch stop, or other separation layer is positioned between the second substrate and the adhesive layer. The silicon substrate is removed to expose the flat surface of a silicon dioxide layer. The second substrate provides an intermediate support prior to transferring the circuit to a releasable third substrate or a flexible application-specific substrate.

The application-specific substrate can be a highly flexible material such as a plastic or Teflon material. The final substrate is prepared to receive the circuit by providing a second layer of adhesive without a separation layer. The circuit is then transferred from the second substrate onto the final substrate such that the flat surface of the silicon dioxide layer bonds with the second adhesive layer. Optionally, the silicon dioxide layer can be further processed to fabricate and/or interconnect devices with the single crystal silicon layer before transfer to the second substrate. The resulting structure is submerged in an acidic solution, such as hydro-flouric (HF) acid, to remove the second substrate such as glass. Such solution provides means for removing the glass and other substrates while rendering the final substrate and separation layers, such as Teflon, copper or amorphous silicon, intact.

Alternatively, in either single or double transfer process, the separation layer itself can have reduced adherence to the substrate which is bonded directly to the adhesive layer along an exposed peripheral region or annular ring around the separation layer. Dicing of the structure serves to release circuits which are then only loosely adhered to the substrate with the separation layer. In fact, the intrinsic stress that develops in the structure during fabrication can cause the structure to bend or delaminate after dicing and can simply be lifted from the substrate.

A copper layer can be used to provide a separation layer between one substrate and the adhesive. Preferably, the separation layer is between 100 521 and 1000 Å in thickness. The thickness of the adhesive layer is preferably less than 75 microns. The overall thickness of a single layer flexible circuit of the present invention is preferably less than 100 microns. Depending on the application, however, the overall thickness can range from 10 to 100 microns.

Another preferred embodiment can utilize the stacking of two or more layers of flexible structures. Each flexible layer can have single or double sided circuit processing and/or can be used to circuit interconnect different layers within the three dimensional circuit structure. The different circuit layers can have differing levels of rigidity such that upon final device fabrication, the resulting laminated circuit structure has a desired level of flexibility. Both single and multilayer circuit devices can be configured to have more flexibility along one axis of the device than one or more other axes of the device. This can be due to the greater flexibility of the circuit elements themselves along a particular axis, or due to the specific application which can require greater flexibility along a particular axis. The size and orientation of the spaces between semiconductive regions of the integrated circuit can be designed to accommodate the difference in flexibility requirements along different axes. These layers can be used to increase or maximize fold endurance of the circuit along one or more selected axes.

Flexible display may be formed from multiple layers of material. These layers may include a touch sensor layer such as a layer on which a pattern of indium tin oxide (ITO) electrodes or other suitable transparent electrodes have been deposited to form a capacitive touch sensor array. These layers may also include a layer that contains an array of display pixels. The touch sensor layer and the display layer may be formed using flexible sheets of polymer or other substrates having thicknesses of 10 microns to 0.5 mm or other suitable thicknesses (as an example).

The display pixel array may be, for example, an organic light-emitting diode (OLED) array. Other types of flexible display pixel arrays may also be formed (e.g., electronic ink displays, etc.). The use of OLED technology to form flexible display is sometimes described herein as an example. This is, however, merely illustrative. Flexible display may be formed using any suitable flexible display technology. The use of flexible displays that are based on OLED technology is merely illustrative.

In addition to these functional display layers (i.e., the OLED array and the optional touch sensor array), display may include one or more structural layers. For example, display may be covered with a flexible or rigid cover layer and/or may be mounted on a support structure (e.g., a rigid support). Layers of adhesive may be used in attaching flexible display layers to each other and may be used in mounting flexible display layers to rigid and flexible structural layers.

In configurations for display in which the cover layer for display is flexible, input-output components that rely on the presence of flexible layers may be mounted at any suitable location under the display (e.g., along peripheral portions of the display, in a central portion of the display, etc.). In configurations for display in which the flexible layers are covered by a rigid cover glass layer or other rigid cover layer, the rigid layer may be provided with one or more openings and the electronic components may be mounted under the openings. Device may also have other openings (e.g., openings in display and/or housing for accommodating volume buttons, ringer buttons, sleep buttons, and other buttons, openings for an audio jack, data port connectors, removable media slots, etc.).

The flexible display may be formed by stacking multiple layers including flexible display layer, touch-sensitive layer, and cover layer. Flexible display may also include other layers of material such as adhesive layers, optical films, or other suitable layers. Flexible display layer 14 may include image pixels formed form light-emitting diodes (LEDs), organic LEDs (OLEDs), plasma cells, electronic ink elements, liquid crystal display (LCD) components, or other suitable image pixel structures compatible with flexible displays.

Touch-sensitive layer may incorporate capacitive touch electrodes such as horizontal transparent electrodes and vertical transparent electrodes. Touch-sensitive layer may, in general, be configured to detect the location of one or more touches or near touches on touch-sensitive layer based on capacitive, resistive, optical, acoustic, inductive, or mechanical measurements, or any phenomena that can be measured with respect to the occurrences of the one or more touches or near touches in proximity to touch sensitive layer.

Software and/or hardware may be used to process the measurements of the detected touches to identify and track one or more gestures. A gesture may correspond to stationary or non-stationary, single or multiple, touches or near touches on touch-sensitive layer 14B. A gesture may be performed by moving one or more fingers or other objects in a particular manner on touch-sensitive layer 14B such as tapping, pressing, rocking, scrubbing, twisting, changing orientation, pressing with varying pressure and the like at essentially the same time, contiguously, or consecutively. A gesture may be characterized by, but is not limited to a pinching, sliding, swiping, rotating, flexing, dragging, or tapping motion between or with any other finger or fingers. A single gesture may be performed with one or more hands, by one or more users, or any combination thereof.

Cover layer may be formed from plastic or glass (sometimes referred to as display cover glass) and may be flexible or rigid. If desired, the interior surface of peripheral inactive portions of cover layer may be provided with an opaque masking layer on such as black ink.

Touch-sensitive flexible display section may be formed from display pixel array layer and optional touch sensor layer. Pressure may cause flexible display to temporarily deform outward of the wearable device such as the watch. Pressure may, if desired, be formed by an internal actuator that deforms display to provide a desired tactile sensation on the surface of display to a user. Flexible display may have a natural resiliency that, following deformation causes flexible display to temporarily deform outward of the watch before returning to its natural shape. Internal component may be a button, an actuator such as a motor, solenoid, vibrator, or piezoelectric actuator, a pressure sensor, an audio component such as a microphone or speaker, or other component. Because display is flexible, these components may operate effectively, even when covered by display. For example, audio components such as microphones and speakers may receive and transmit sound through flexible display. A barometric pressure sensor or a force sensor may also receive input through flexible display. Components such as actuators may be used to temporarily create raised ridges or other external features on the surface of the flexible display (e.g., to indicate to a user where an on-screen button or group of buttons is located).

In one embodiment, touch-sensitive flexible display section may be deformed to depress a dome switch, thereby activating the switch (e.g., shorting internal switch terminals together to close the switch). In these embodiments, the flexible display can form an array of dome switches that provide tactile feedback to typists. The use of a dome switch with a dome-shaped biasing structure is merely illustrative and other switch shapes such as rectangular shaped like a keyboard switch can be done.

When an active display portion is configured so as to overlap buttons and other components, there is generally more area available for the active display portion. The presence of flexible display over button may also reduce the risk of moisture or dirt entering into the interior of device.

An actuator such as a piezoelectric actuator can be used to detect actuation and/or provide tactile feedback. The actuator may vary in shape (e.g., thickness) in response to applied control voltages and may produce an output voltage when compressed (i.e., the piezoelectric element in actuator may serve as a force sensor in addition to serving as a controllable actuator). A user may exert force on flexible display in a direction. Flexible display may be deformed to exert a mechanical pressure on piezoelectric element or other force sensor, inducing a voltage which may be transmitted to the wearable device. Conversely, piezoelectric actuator may be used to provide tactile feedback to a. A voltage difference applied to the surfaces of piezoelectric actuator may induce an expansion of piezoelectric actuator 90. Piezoelectric actuator may then deform flexible display for providing tactile feedback to a user.

A touch sensor array associated with display may be used to gather user input (i.e., the touch sensor array may be used to determine when a user has pressed the virtual key associated with portion). The location of portion may also be indicated visually using associated display pixels in flexible display. At times, a user may desire to be able to locate portion without having to look at flexible display. Deforming flexible display in the vicinity of portion using structural component may allow a user to locate portion without visual aid. Structural component may be an isolated component indicating the location of a single portion of touch-sensitive layer or may be one of an array of components indicating the locations of an array of portions (e.g., the array of letter, number, and symbol keys in a virtual keypad displayed on display). Structural component may be a separate component mounted to support structures or may be an integral part of support structures. The location of interface component may be indicated visually using display pixels in flexible display. The deformation of flexible display in the vicinity of interface component using structural component may also allow the user to locate interface component without visual aid. A ridge or other deformation such as deformation in flexible display may be used to indicate the location of button.

The watch can be configured to be coupled to the wrist band 102 including a flexible battery integrated therein. While the flexible battery described herein can be flexible lithium, prismatic, or cylindrical battery cell. It is contemplated that such a battery is fabricated with flexible material which is further adapted to function as the wrist band 102 or otherwise as a belt for attaching the watch 100 to a user.

The wrist band 102 includes a battery portion 106 to integrate the battery therein. Accordingly, positive and negative portions of the battery portion 106 are electrically coupled to the watch 100. The terminals are also adapted to electrically and mechanically couple the battery to a charging device. FIG. 2 shows another perspective view of the wearable watch 100 with the wrist 102 band having the flexible battery unit integrally formed therein, according to embodiments described herein.

FIG. 4C shows a perspective view of the flexible battery unit 300, according to embodiments described herein. The flexible battery unit 300 can be configured to be connected to the control circuit electrically. In the third embodiment, the flexible battery unit 300 may be provided with the flexibility through the interconnection of individual elements. The flexible battery unit 300 can include a plurality of battery flexible sub-units 301 connecting flexible circuit elements, which are disposed between the battery flexible sub-units 301 respectively to connect the battery flexible sub-units 301 physically and electrically.

In one embodiment, the flexible electronic controller further includes a power supply which is constructed and arranged to provide power to the flexible electronic circuitry 34. In some arrangements, the power supply generates power (e.g., due to a chemical reaction). In some arrangements, the power supply stores power from a main power feed (e.g., a rechargeable battery storing a charge). Gel, chemicals, or physical batteries are examples of power supplies which are capable of being used in these arrangements. In some arrangements, the power supply derives power from the environment, e.g., the power supply includes photovoltaic cells that respond to light and/or power generators that respond to heat and/or vibration.

FIG. 4D is a diagram 400 illustrating generally, communication between wearable watch 100 and smart phone 402, according to embodiments described herein. In an embodiment, the wearable watch 120 can be configured to communicate with the smart phone 402 of the user. In an embodiment, the smart phone 402 described herein can be a tablet, computer, communicator, mobile phone, or any other electronic device. The wearable watch 120 can be configured to include sufficient interfaces and firmware to communicate with the smart phone 402. The flexible battery unit can be configured to provide power and controlled through the control circuit, such as to communicate with the smart phone 402.

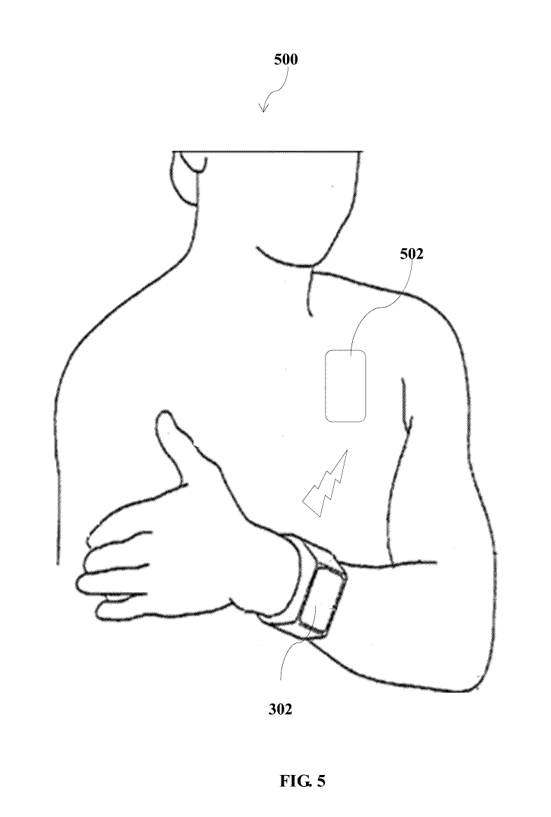

FIG. 5 is a diagram 500 illustrating generally, communication between the wearable watch 100 and implantable device 502, according to embodiments described herein. One embodiment, the FIG. 5 includes bioelectrical impedance (BI) spectroscopy sensors in addition to or as alternates to EKG sensors and heart sound transducer sensors. BI spectroscopy is based on Ohm's Law: current in a circuit is directly proportional to voltage and inversely proportional to resistance in a DC circuit or impedance in an alternating current (AC) circuit. Bioelectric impedance exchanges electrical energy with the patient body or body segment. The exchanged electrical energy can include alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz. A BI instrument operating at the single frequency of 50 KHz reflects primarily the extra cellular water compartment as a very small current passes through the cell. Because low frequency (<1 KHz) current does not penetrate the cells and that complete penetration occurs only at a very high frequency (>1 MHz), multi-frequency BI or bioelectrical impedance spectroscopy devices can be used to scan a wide range of frequencies.

In a tetra polar implementation, two electrodes on the wrist watch or wrist band are used to apply AC or DC constant current into the body or body segment. The voltage signal from the surface of the body is measured in terms of impedance using the same or an additional two electrodes on the watch or wrist band. In a bipolar implementation, one electrode on the wrist watch or wrist band is used to apply AC or DC constant current into the body or body segment. The voltage signal from the surface of the body is measured in terms of impedance using the same or an alternative electrode on the watch or wrist band. The system of FIG. 5 may include the implantable device 502 that wirelessly communicates BI information with the wrist watch. In an embodiment, other electronic devices or patches can be used to collect other medical information or vital parameter and communicate with the wrist watch or base station or the information could be relayed through each wireless node or appliance to reach a destination appliance such as the base station, for example.

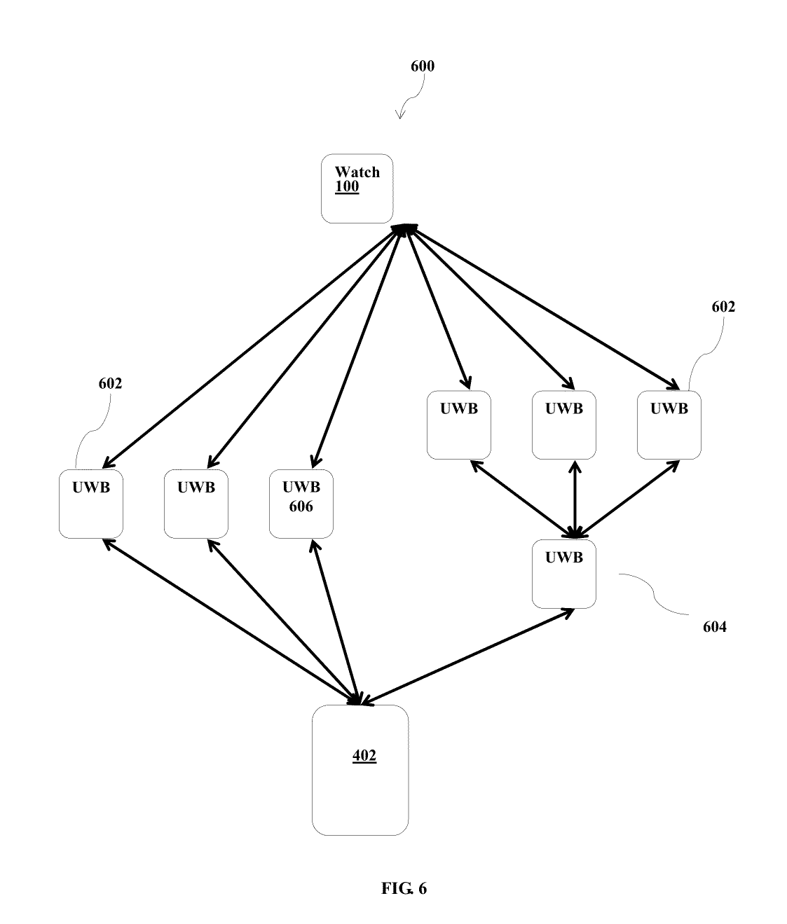

FIG. 6 shows a graphical overview of wearable watch system 600, according to embodiments described herein. In an embodiment, the watch device 100 (one shown at the top of the diagram) communicate with the rest of the infrastructure through for example, 802.15.4 radio protocol, or any other protocol. The watch 100 can be configured to associate with any base station 604 in the system 100, with preference given to nearby base stations (based on signal strength and link quality). For example, communication to and from the watch 100 can be routed wirelessly to a base station that is connected to a computer, running the radio master program, which distributes the received radio messages to various other parts of the software system. In an embodiment, ultra-wide band (UWB) 606 tracking tag worn alongside the watch 100 can be configured to emit UWB pulses that are received by the smart phone 402 software, which sends a list of tag locations to the watch software through, for example, UDP/IP messages.

Various exemplary glanceable screens can be shown on the watch 100, according to embodiments as disclosed herein. In an embodiment, services provided by the watch-based system include real-time stock quotes, stock trading, weather updates, traffic alerts, sports scores, flight confirmation, news flashes, currency conversion, online yellow pages, games, mobile banking, mobile stock trading and other location-based, time-sensitive information.

In an embodiment, the watch 100 can be configured to include a series of buttons or scrollable keypads which are arranged to operate as part of a user interface (UI). Each button may have a default function and/or a context determined function. The currently selected channel determines the context for each button. Alternatively, the currently active display may determine the context for each button. For example, a display screen (e.g., a help screen) may be superimposed on the main display such that the display screen becomes the active context. In an embodiment, the watch 400 can be context sensitive in that the function that is associated with each button may change based on the selected channel or display screen. For example, button “A” has a default function of page up or previous page in the currently selected channel. Button “A” may also have an alternate function based on the currently selected channel or display. For example, button “A” may be configured to activate a speed list browse function after button “A” is activated for a predetermined time interval. In the speed list browse function, a pop-up visual cue (e.g., a pop-up window) may be used to indicate how that list is indexed. Button “B” has a default function of page down or next page in the currently selected channel. The button “B” may also have an alternate function based on the currently selected channel or display. In one example, button “B” is activated for a predetermined time interval (e.g., two seconds) to select a “speed list browse” function. Button “C” has a default function of next channel. The button “C” may also have an alternate function based on the currently selected channel or display. In one example, button “C” is activated for a predetermined time interval (e.g., two seconds) to select the main channel or “primary” channel. The main channel in an example device can be a news channel that provides the user with fresh news and information. However, devices may be configured to have some other display screen that is recognized by the device as a “primary” channel or “home” location. Button “D” has a default (or “primary”) function of “enter.” The “enter” function is context sensitive and used to select the “enter” function within a selected channel, or to select an item from a selection list. The button “D” may also have an alternate function based on the currently selected channel or display. For example, the “D” button is activated for a predetermined time interval (e.g., two seconds) to activate a delete function. In another example, the “D” button may be selected for a predetermined time to activate a help screen or an additional set mode. In this example, the help screen remains active while button “D” is activated, and the help screen is deactivated (e.g., removed from the display) when the “D” button is released. The buttons are arranged such that the electronic device accomplishes navigating and selecting content on each channel in a simple manner. An optional fifth button (e.g., button “E”) may be arranged to provide other functions such as backlighting or another desired function. Other buttons may also be included.

Data is collected and communicated on the display of the wearable device. In one embodiment, voice is transmitted to a base station for communicating over a network to an authorized party. The wearable device and the base station is part of a mesh network that may communicate with a medicine cabinet to detect opening or to each medicine container to detect medication compliance. Other devices include mesh network thermometers, scales, or exercise devices. The mesh network also includes a plurality of home/room appliances. The ability to transmit voice is useful in the case the patient has fallen down and cannot walk to the base station 1390 to request help. Hence, in one embodiment, the wearable device captures voice from the user and transmits the voice over the Zigbee mesh network to the base station. The base station 1390 in turn dials out to an authorized third party to allow voice communication and at the same time transmits the collected patient vital parameter data and identifying information so that help can be dispatched quickly, efficiently and error-free. In one embodiment, the base station is a POTS telephone base station connected to the wired phone network. In a second embodiment, the base station can be a cellular telephone connected to a cellular network for voice and data transmission. In a third embodiment, the base station can be a WiMAX or 802.16 standard base station that can communicate VOIP and data over a wide area network. I one implementation, Zigbee or 802.15 appliances communicate locally and then transmits to the wide area network (WAN) such as the Internet over WiFi or WiMAX. Alternatively, the base station can communicate with the WAN over POTS and a wireless network such as cellular or WiMAX or both. In another embodiment, Bluetooth

The digital device 120 or wearable device includes bioelectrical impedance (BI) spectroscopy sensors in addition to or as alternates to EKG sensors and heart sound transducer sensors. BI spectroscopy is based on Ohm's Law: current in a circuit is directly proportional to voltage and inversely proportional to resistance in a DC circuit or impedance in an alternating current (AC) circuit. Bioelectric impedance exchanges electrical energy with the patient body or body segment. The exchanged electrical energy can include alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz. A BI instrument operating at the single frequency of 50 KHz reflects primarily the extra cellular water compartment as a very small current passes through the cell. Because low frequency (<1 KHz) current does not penetrate the cells and that complete penetration occurs only at a very high frequency (>1 MHz), multi-frequency BI or bioelectrical impedance spectroscopy devices can be used to scan a wide range of frequencies.

The digial wearable device 120 includes a health monitoring system. The system can operate in a home, a nursing home, or a hospital. In this system, one or more mesh network appliances are provided to enable wireless communication in the home monitoring system. Appliances in the mesh network can include home security monitoring devices, door alarm, window alarm, home temperature control devices, fire alarm devices, among others. Appliances in the mesh network can be one of multiple portable physiological transducer, such as a blood pressure monitor, heart rate monitor, weight scale, thermometer, spirometer, single or multiple lead electrocardiograph (ECG), a pulse oxymeter, a body fat monitor, a cholesterol monitor, a signal from a medicine cabinet, a signal from a drug container, a signal from a commonly used appliance such as a refrigerator/stove/oven/washer, or a signal from an exercise machine, such as a heart rate. As will be discussed in more detail below, one appliance is a patient monitoring device that can be worn by the patient and includes a single or bi-directional wireless communication link, generally identified by the bolt symbol in for transmitting data from the appliances to the local hub or receiving station or base station server by way of a wireless radio frequency (RF) link using a proprietary or non-proprietary protocol. For example, within a house, a user may have mesh network appliances that detect window and door contacts, smoke detectors and motion sensors, video cameras, key chain control, temperature monitors, CO and other gas detectors, vibration sensors, and others. A user may have flood sensors and other detectors on a boat. An individual, such as an ill or elderly grandparent, may have access to a panic transmitter or other alarm transmitter. Other sensors and/or detectors may also be included. The user may register these appliances on a central security network by entering the identification code for each registered appliance/device and/or system. The mesh network can be Zigbee network or 802.15 network.

The patient may wear one or more wearable patient monitoring appliances such as wrist-wearable devicees or clip on devices or electronic jewelry to monitor the patient. One wearable appliance such as a wrist-wearable device includes sensors, for example devices for sensing ECG, EKG, blood pressure, sugar level, among others. In one embodiment, the sensors are mounted on the patient's wrist (such as a wristwearable device sensor) and other convenient anatomical locations. Exemplary sensors include standard medical diagnostics for detecting the body's electrical signals emanating from muscles (EMG and EOG) and brain (EEG) and cardiovascular system (ECG). Leg sensors can include piezoelectric accelerometers designed to give qualitative assessment of limb movement. Additionally, thoracic and abdominal bands used to measure expansion and contraction of the thorax and abdomen respectively. A small sensor can be mounted on the subject's finger in order to detect blood-oxygen levels and pulse rate. Additionally, a microphone can be attached to throat and used in sleep diagnostic recordings for detecting breathing and other noise. One or more position sensors can be used for detecting orientation of body (lying on left side, right side or back) during sleep diagnostic recordings. Each of sensors can individually transmit data to the server using wired or wireless transmission. Alternatively, all sensors can be fed through a common bus into a single transceiver for wired or wireless transmission. The transmission can be done using a magnetic medium such as a floppy disk or a flash memory card, or can be done using infrared or radio network link, among others. The sensor can also include an indoor positioning system or alternatively a global position system (GPS) receiver that relays the position and ambulatory patterns of the patient to the server for mobility tracking.

In one embodiment, the sensors for monitoring vital signs are enclosed in a wrist-wearable device sized case supported on a wrist band. The sensors can be attached to the back of the case. For example, in one embodiment, Cygnus' AutoSensor (Redwood City, Calif.) is used as a glucose sensor. A low electric current pulls glucose through the skin. Glucose is accumulated in two gel collection discs in the AutoSensor. The AutoSensor measures the glucose and a reading is displayed by the wearable device.

In another embodiment, EKG/ECG contact points are positioned on the back of the wrist-wearable device case. In yet another embodiment that provides continuous, beat-to-beat wrist arterial pulse rate measurements, a pressure sensor is housed in a casing with a ‘free-floating’ plunger as the sensor applanates the radial artery. A strap provides a constant force for effective applanation and ensuring the position of the sensor housing to remain constant after any wrist movements. The change in the electrical signals due to change in pressure is detected as a result of the piezoresistive nature of the sensor are then analyzed to arrive at various arterial pressure, systolic pressure, diastolic pressure, time indices, and other blood pressure parameters.

The case may be of a number of variations of shape but can be conveniently made a rectangular, approaching a box-like configuration. The wrist-band can be an expansion band or a wristwearable device strap of plastic, leather or woven material. The wrist-band further contains an antenna for transmitting or receiving radio frequency signals. The wristband and the antenna inside the band are mechanically coupled to the top and bottom sides of the wrist-wearable device housing. Further, the antenna is electrically coupled to a radio frequency transmitter and receiver for wireless communications with another computer or another user. Although a wrist-band is disclosed, a number of substitutes may be used, including a belt, a ring holder, a brace, or a bracelet, among other suitable substitutes known to one skilled in the art. The housing contains the processor and associated peripherals to provide the human-machine interface. A display is located on the front section of the housing. A speaker, a microphone, and a plurality of push-button switches and are also located on the front section of housing. An infrared LED transmitter and an infrared LED receiver are positioned on the right side of housing to enable the wearable device to communicate with another computer using infrared transmission.

Sensors can be mounted on fixed surfaces such as walls or tables, for example. One such sensor is a motion detector. Another sensor is a proximity sensor. The fixed sensors can operate alone or in conjunction with the cameras. In one embodiment where the motion detector operates with the cameras, the motion detector can be used to trigger camera recording. Thus, as long as motion is sensed, images from the cameras are not saved. However, when motion is not detected, the images are stored and an alarm may be generated. In another embodiment where the motion detector operates stand alone, when no motion is sensed, the system generates an alarm.

A server also executes one or more software modules to analyze data from the patient. A module 50 monitors the patient's vital signs such as ECG/EKG and generates warnings should problems occur. In this module, vital signs can be collected and communicated to the server using wired or wireless transmitters. In one embodiment, the server feeds the data to a statistical analyzer such as a neural network which has been trained to flag potentially dangerous conditions. The neural network can be a back-propagation neural network, for example. In this embodiment, the statistical analyzer is trained with training data where certain signals are determined to be undesirable for the patient, given his age, weight, and physical limitations, among others. For example, the patient's glucose level should be within a well established range, and any value outside of this range is flagged by the statistical analyzer as a dangerous condition. As used herein, the dangerous condition can be specified as an event or a pattern that can cause physiological or psychological damage to the patient. Moreover, interactions between different vital signals can be accounted for so that the statistical analyzer can take into consideration instances where individually the vital signs are acceptable, but in certain combinations, the vital signs can indicate potentially dangerous conditions. Once trained, the data received by the server can be appropriately scaled and processed by the statistical analyzer. In addition to statistical analyzers, the server can process vital signs using rule-based inference engines, fuzzy logic, as well as conventional if-then logic. Additionally, the server can process vital signs using Hidden Markov Models (HMMs), dynamic time warping, or template matching, among others.

The patient may wear one or more wearable patient monitoring appliances such as wrist-wearable devicees or clip on devices or electronic jewelry to monitor the patient. One wearable appliance such as a wrist-wearable device includes sensors, for example devices for sensing ECG, EKG, blood pressure, sugar level, among others. In one embodiment, the sensors are mounted on the patient's wrist (such as a wristwearable device sensor) and other convenient anatomical locations. Exemplary sensors include standard medical diagnostics for detecting the body's electrical signals emanating from muscles (EMG and EOG) and brain (EEG) and cardiovascular system (ECG). Leg sensors can include piezoelectric accelerometers designed to give qualitative assessment of limb movement. Additionally, thoracic and abdominal bands used to measure expansion and contraction of the thorax and abdomen respectively.

A small sensor can be mounted on the subject's finger in order to detect blood-oxygen levels and pulse rate. Additionally, a microphone can be attached to throat and used in sleep diagnostic recordings for detecting breathing and other noise. One or more position sensors can be used for detecting orientation of body (lying on left side, right side or back) during sleep diagnostic recordings. Each of sensors can individually transmit data to the server using wired or wireless transmission. Alternatively, all sensors can be fed through a common bus into a single transceiver for wired or wireless transmission. The transmission can be done using a magnetic medium such as a floppy disk or a flash memory card, or can be done using infrared or radio network link, among others. The sensor can also include an indoor positioning system or alternatively a global position system (GPS) receiver that relays the position and ambulatory patterns of the patient to the server for mobility tracking.

In one embodiment, the sensors for monitoring vital signs are enclosed in a wrist-wearable device sized case supported on a wrist band. The sensors can be attached to the back of the case. For example, in one embodiment, Cygnus' AutoSensor (Redwood City, Calif.) is used as a glucose sensor. A low electric current pulls glucose through the skin. Glucose is accumulated in two gel collection discs in the AutoSensor. The AutoSensor measures the glucose and a reading is displayed by the wearable device.

In another embodiment, EKG/ECG contact points are positioned on the back of the wrist-wearable device case. In yet another embodiment that provides continuous, beat-to-beat wrist arterial pulse rate measurements, a pressure sensor is housed in a casing with a ‘free-floating’ plunger as the sensor applanates the radial artery. A strap provides a constant force for effective applanation and ensuring the position of the sensor housing to remain constant after any wrist movements. The change in the electrical signals due to change in pressure is detected as a result of the piezoresistive nature of the sensor are then analyzed to arrive at various arterial pressure, systolic pressure, diastolic pressure, time indices, and other blood pressure parameters. A unit can authenticate the user based on ECG, EMG, EEG, or bioimpedance data. Alternatively, a unit can detect a dangerous condition for the user based on ECG or bioimpedance data. This can be done using neural network or HMMs as detailed below.

The case may be of a number of variations of shape but can be conveniently made a rectangular, approaching a box-like configuration. The wrist-band can be an expansion band or a wristwearable device strap of plastic, leather or woven material. The wrist-band further contains an antenna for transmitting or receiving radio frequency signals. The wristband and the antenna inside the band are mechanically coupled to the top and bottom sides of the wrist-wearable device housing. Further, the antenna is electrically coupled to a radio frequency transmitter and receiver for wireless communications with another computer or another user. Although a wrist-band is disclosed, a number of substitutes may be used, including a belt, a ring holder, a brace, or a bracelet, among other suitable substitutes known to one skilled in the art. The housing contains the processor and associated peripherals to provide the human-machine interface. A display is located on the front section of the housing. A speaker, a microphone, and a plurality of push-button switches and are also located on the front section of housing. An infrared LED transmitter and an infrared LED receiver are positioned on the right side of housing to enable the wearable device to communicate with another computer using infrared transmission.

In another embodiment, the sensors are mounted on the patient's clothing. For example, sensors can be woven into a single-piece garment (an undershirt) on a weaving machine. A plastic optical fiber can be integrated into the structure during the fabric production process without any discontinuities at the armhole or the seams. An interconnection technology transmits information from (and to) sensors mounted at any location on the body thus creating a flexible “bus” structure. T-Connectors—similar to “button clips” used in clothing—are attached to the fibers that serve as a data bus to carry the information from the sensors (e.g., EKG sensors) on the body. The sensors will plug into these connectors and at the other end similar T-Connectors will be used to transmit the information to monitoring equipment or personal status monitor. Since shapes and sizes of humans will be different, sensors can be positioned on the right locations for all patients and without any constraints being imposed by the clothing. Moreover, the clothing can be laundered without any damage to the sensors themselves. In addition to the fiber optic and specialty fibers that serve as sensors and data bus to carry sensory information from the wearer to the monitoring devices, sensors for monitoring the respiration rate can be integrated into the structure.

In another embodiment, instead of being mounted on the patient, the sensors can be mounted on fixed surfaces such as walls or tables, for example. One such sensor is a motion detector. Another sensor is a proximity sensor. The fixed sensors can operate alone or in conjunction with the cameras 10. In one embodiment where the motion detector operates with the cameras 10, the motion detector can be used to trigger camera recording. Thus, as long as motion is sensed, images from the cameras 10 are not saved. However, when motion is not detected, the images are stored and an alarm may be generated. In another embodiment where the motion detector operates stand alone, when no motion is sensed, the system generates an alarm.

The server also executes one or more software modules to analyze data from the patient. A module 50 monitors the patient's vital signs such as ECG/EKG and generates warnings should problems occur. In this module, vital signs can be collected and communicated to the server using wired or wireless transmitters. In one embodiment, the server feeds the data to a statistical analyzer such as a neural network which has been trained to flag potentially dangerous conditions. The neural network can be a back-propagation neural network, for example. In this embodiment, the statistical analyzer is trained with training data where certain signals are determined to be undesirable for the patient, given his age, weight, and physical limitations, among others. For example, the patient's glucose level should be within a well established range, and any value outside of this range is flagged by the statistical analyzer as a dangerous condition. As used herein, the dangerous condition can be specified as an event or a pattern that can cause physiological or psychological damage to the patient. Moreover, interactions between different vital signals can be accounted for so that the statistical analyzer can take into consideration instances where individually the vital signs are acceptable, but in certain combinations, the vital signs can indicate potentially dangerous conditions. Once trained, the data received by the server can be appropriately scaled and processed by the statistical analyzer. In addition to statistical analyzers, the server can process vital signs using rule-based inference engines, fuzzy logic, as well as conventional if-then logic. Additionally, the server can process vital signs using Hidden Markov Models (HMMs), dynamic time warping, or template matching, among others.

Data collected and communicated on the display of the wearable device as well as voice is transmitted to a base station for communicating over a network to an authorized party. The wearable device and the base station is part of a mesh network that may communicate with a medicine cabinet to detect opening or to each medicine container to detect medication compliance. Other devices include mesh network thermometers, scales, or exercise devices. The mesh network also includes a plurality of home/room appliances. The ability to transmit voice is useful in the case the patient has fallen down and cannot walk to the base station to request help. Hence, in one embodiment, the wearable device captures voice from the user and transmits the voice over the Zigbee mesh network to the base statio. The base station in turn dials out to an authorized third party to allow voice communication and at the same time transmits the collected patient vital parameter data and identifying information so that help can be dispatched quickly, efficiently and error-free. In one embodiment, the base station is a POTS telephone base station connected to the wired phone network. In a second embodiment, the base station can be a cellular telephone connected to a cellular network for voice and data transmission. In a third embodiment, the base station can be a WiMAX or 802.16 standard base station that can communicate VOIP and data over a wide area network. I one implementation, Zigbee or 802.15 appliances communicate locally and then transmits to the wide area network (WAN) such as the Internet over WiFi or WiMAX. Alternatively, the base station can communicate with the WAN over POTS and a wireless network such as cellular or WiMAX or both.

The wearable device includes bioelectrical impedance (BI) spectroscopy sensors in addition to or as alternates to EKG sensors and heart sound transducer sensors. BI spectroscopy is based on Ohm's Law: current in a circuit is directly proportional to voltage and inversely proportional to resistance in a DC circuit or impedance in an alternating current (AC) circuit. Bioelectric impedance exchanges electrical energy with the patient body or body segment. The exchanged electrical energy can include alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz. A BI instrument operating at the single frequency of 50 KHz reflects primarily the extra cellular water compartment as a very small current passes through the cell. Because low frequency (<1 KHz) current does not penetrate the cells and that complete penetration occurs only at a very high frequency (>1 MHz), multi-frequency BI or bioelectrical impedance spectroscopy devices can be used to scan a wide range of frequencies.

In a tetrapolar implementation, two electrodes on the wrist wearable device or wrist band are used to apply AC or DC constant current into the body or body segment. The voltage signal from the surface of the body is measured in terms of impedance using the same or an additional two electrodes on the wearable device or wrist band. In a bipolar implementation, one electrode on the wrist wearable device or wrist band is used to apply AC or DC constant current into the body or body segment. The voltage signal from the surface of the body is measured in terms of impedance using the same or an alternative electrode on the wearable device or wrist band. The system may include a BI patch that wirelessly communicates BI information with the wrist wearable device. Other patches can be used to collect other medical information or vital parameter and communicate with the wrist wearable device or base station or the information could be relayed through each wireless node or appliance to reach a destination appliance such as the base station, for example. The system can also include a head-cap that allows a number of EEG probes access to the brain electrical activities, EKG probes to measure cranial EKG activity, as well as BI probes to determine cranial fluid presence indicative of a stroke. As will be discussed below, the EEG probes allow the system to determine cognitive status of the patient to determine whether a stroke had just occurred, the EKG and the BI probes provide information on the stroke to enable timely treatment to minimize loss of functionality to the patient if treatment is delayed.

Bipolar or tetrapolar electrode systems can be used in the BI instruments. Of these, the tetrapolar system provides a uniform current density distribution in the body segment and measures impedance with less electrode interface artifact and impedance errors. In the tetrapolar system, a pair of surface electrodes is used as current electrodes to introduce a low intensity constant current at high frequency into the body. A pair of electrodes measures changes accompanying physiological events. Voltage measured across is directly proportional to the segment electrical impedance of the human subject. Circular flat electrodes as well as band type electrodes can be used. In one embodiment, the electrodes are in direct contact with the skin surface. In other embodiments, the voltage measurements may employ one or more contactless, voltage sensitive electrodes such as inductively or capacitively coupled electrodes. The current application and the voltage measurement electrodess in these embodiments can be the same, adjacent to one another, or at significantly different locations. The electrode(s) can apply current levels from uA to 10 mA rms at a frequency range of −100 KHz. A constant current source and high input impedance circuit is used in conjunction with the tetrapolar electrode configuration to avoid the contact pressure effects at the electrode-skin interface.

The BI sensor can be a Series Model which assumes that there is one conductive path and that the body consists of a series of resistors. An electrical current, injected at a single frequency, is used to measure whole body impedance (i.e., wrist to ankle) for the purpose of estimating total body water and fat free mass. Alternatively, the BI instrument can be a Parallel BI Model In this model of impedance, the resistors and capacitors are oriented both in series and in parallel in the human body. Whole body BI can be used to estimate TBW and FFM in healthy subjects or to estimate intracellular water (ICW) and body cell mass (BCM). High-low BI can be used to estimate extracellular water (ECW) and total body water (TBW). Multi-frequency BI can be used to estimate ECW, ICW, and TBW; to monitor changes in the ECW/BCM and ECW/TBW ratios in clinical populations. The instrument can also be a Segmental BI Model and can be used in the evaluation of regional fluid changes and in monitoring extra cellular water in patients with abnormal fluid distribution, such as those undergoing hemodialysis. Segmental BI can be used to measure fluid distribution or regional fluid accumulation in clinical populations. Upper-body and Lower-body BI can be used to estimate percentage BF in healthy subjects with normal hydration status and fluid distribution. The BI sensor can be used to detect acute dehydration, pulmonary edema (caused by mitral stenosis or left ventricular failure or congestive heart failure, among others), or hyper-hydration cause by kidney dialysis, for example. In one embodiment, the system determines the impedance of skin and subcutaneous adipose tissue using tetrapolar and bipolar impedance measurements. In the bipolar arrangement the inner electrodes act both as the electrodes that send the current (outer electrodes in the tetrapolar arrangement) and as receiving electrodes. If the outer two electrodes (electrodes sending current) are superimposed onto the inner electrodes (receiving electrodes) then a bipolar BIA arrangement exists with the same electrodes acting as receiving and sending electrodes. The difference in impedance measurements between the tetrapolar and bipolar arrangement reflects the impedance of skin and subcutaneous fat. The difference between the two impedance measurements represents the combined impedance of skin and subcutaneous tissue at one or more sites. The system determines the resistivities of skin and subcutaneous adipose tissue, and then calculates the skinfold thickness (mainly due to adipose tissue).

Various BI analysis methods can be used in a variety of clinical applications such as to estimate body composition, to determine total body water, to assess compartmentalization of body fluids, to provide cardiac monitoring, measure blood flow, dehydration, blood loss, wound monitoring, ulcer detection and deep vein thrombosis. Other uses for the BI sensor includes detecting and/or monitoring hypovolemia, hemorrhage or blood loss. The impedance measurements can be made sequentially over a period of in time; and the system can determine whether the subject is externally or internally bleeding based on a change in measured impedance. The wearable device can also report temperature, heat flux, vasodilation and blood pressure along with the BI information.

In one embodiment, the BI system monitors cardiac function using impedance cardiography (ICG) technique. ICG provides a single impedance tracing, from which parameters related to the pump function of the heart, such as cardiac output (CO), are estimated. ICG measures the beat-to-beat changes of thoracic bioimpedance via four dual sensors applied on the neck and thorax in order to calculate stroke volume (SV). By using the resistivity ρ of blood and the length L of the chest, the impedance change ΔZ and base impedance (Zo) to the volume change ΔV of the tissue under measurement can be derived as follows:

The impedance cardiographic embodiment allows hemodynamic assessment to be regularly monitored to avoid the occurrence of an acute cardiac episode. The system provides an accurate, noninvasive measurement of cardiac output (CO) monitoring so that ill and surgical patients undergoing major operations such as coronary artery bypass graft (CABG) would benefit. In addition, many patients with chronic and comorbid diseases that ultimately lead to the need for major operations and other costly interventions might benefit from more routine monitoring of CO and its dependent parameters such as systemic vascular resistance (SVR).

In one embodiment to monitor heart failure, an array of BI sensors are place in proximity to the heart. The array of BI sensors detect the presence or absence, or rate of change, or body fluids proximal to the heart. The BI sensors can be supplemented by the EKG sensors. A normal, healthy, heart beats at a regular rate. Irregular heartbeats, known as cardiac arrhythmia, on the other hand, may characterize an unhealthy condition. Another unhealthy condition is known as congestive heart failure (“CHF”). CHF, also known as heart failure, is a condition where the heart has inadequate capacity to pump sufficient blood to meet metabolic demand. CHF may be caused by a variety of sources, including, coronary artery disease, myocardial infarction, high blood pressure, heart valve disease, cardiomyopathy, congenital heart disease, endocarditis, myocarditis, and others. Unhealthy heart conditions may be treated using a cardiac rhythm management (CRM) system. Examples of CRM systems, or pulse generator systems, include defibrillators (including implantable cardioverter defibrillator), pacemakers and other cardiac resynchronization devices.

In one implementation, BIA measurements can be made using an array of bipolar or tetra polar electrodes that deliver a constant alternating current at 50 KHz frequency. Whole body measurements can be done using standard right-sided. The ability of any biological tissue to resist a constant electric current depends on the relative proportions of water and electrolytes it contains, and is called resistivity (in Ohms/cm 3). The measuring of bioimpedance to assess congestive heart failure employs the different bio-electric properties of blood and lung tissue to permit separate assessment of: (a) systemic venous congestion via a low frequency or direct current resistance measurement of the current path through the right ventricle, right atrium, superior vena cava, and subclavian vein, or by computing the real component of impedance at a high frequency, and (b) pulmonary congestion via a high frequency measurement of capacitive impedance of the lung. The resistance is impedance measured using direct current or alternating current (AC) which can flow through capacitors.

In one embodiment, a belt is worn by the patient with a plurality of BI probes positioned around the belt perimeter. The output of the tetrapolar probes is processed using a second-order Newton-Raphson method to estimate the left and right-lung resistivity values in the thoracic geometry. The locations of the electrodes are marked. During the measurements procedure, the belt is worn around the patient's thorax while sitting, and the reference electrode is attached to his waist. The data is collected during tidal respiration to minimize lung resistivity changes due to breathing, and lasts approximately one minute. The process is repeated periodically and the impedance trend is analyzed to detect CHF. Upon detection, the system provides vital parameters to a call center and the call center can refer to a physician for consultation or can call 9-1-1 for assistance.

In one embodiment, an array of noninvasive thoracic electrical bioimpedance monitoring probes can be used alone or in conjunction with other techniques such as impedance cardiography (ICG) for early comprehensive cardiovascular assessment and trending of acute trauma victims. This embodiment provides early, continuous cardiovascular assessment to help identify patients whose injuries were so severe that they were not likely to survive. This included severe blood and/or fluid volume deficits induced by trauma, which did not respond readily to expeditious volume resuscitation and vasopressor therapy. One exemplary system monitors cardiorespiratory variables that served as statistically significant measures of treatment outcomes: Qt, BP, pulse oximetry, and transcutaneous Pot (Ptco2). A high Qt may not be sustainable in the presence of hypovolemia, acute anemia, pre-existing impaired cardiac function, acute myocardial injury, or coronary ischemia. Thus a fall in Ptco2 could also be interpreted as too high a metabolic demand for a patient's cardiovascular reserve. Too high a metabolic demand may compromise other critical organs. Acute lung injury from hypotension, blunt trauma, and massive fluid resuscitation can drastically reduce respiratory reserve.

One embodiment that measures thoracic impedance (a resistive or reactive impedance associated with at least a portion of a thorax of a living organism). The thoracic impedance signal is influenced by the patient's thoracic intravascular fluid tension, heart beat, and breathing (also referred to as “respiration” or “ventilation”). A “de” or “baseline” or “low frequency” component of the thoracic impedance signal (e.g., less than a cutoff value that is approximately between 0.1 Hz and 0.5 Hz, inclusive, such as, for example, a cutoff value of approximately 0.1 Hz) provides information about the subject patient's thoracic fluid tension, and is therefore influenced by intravascular fluid shifts to and away from the thorax. Higher frequency components of the thoracic impedance signal are influenced by the patient's breathing (e.g., approximately between 0.05 Hz and 2.0 Hz inclusive) and heartbeat (e.g., approximately between 0.5 Hz and 10 Hz inclusive). A low intravascular fluid tension in the thorax (“thoracic hypotension”) may result from changes in posture. For example, in a person who has been in a recumbent position for some time, approximately ⅓ of the blood volume is in the thorax. When that person then sits upright, approximately ⅓ of the blood that was in the thorax migrates to the lower body. This increases thoracic impedance. Approximately 90% of this fluid shift takes place within 2 to 3 minutes after the person sits upright.

The accelerometer can be used to provide reproducible measurements. Body activity will increase cardiac output and also change the amount of blood in the systemic venous system or lungs. Measurements of congestion may be most reproducible when body activity is at a minimum and the patient is at rest. The use of an accelerometer allows one to sense both body position and body activity. Comparative measurements over time may best be taken under reproducible conditions of body position and activity. Ideally, measurements for the upright position should be compared as among themselves. Likewise measurements in the supine, prone, left lateral decubitus and right lateral decubitus should be compared as among themselves. Other variables can be used to permit reproducible measurements, i.e. variations of the cardiac cycle and variations in the respiratory cycle. The ventricles are at their most compliant during diastole. The end of the diastolic period is marked by the QRS on the electrocardiographic means (EKG) for monitoring the cardiac cycle. The second variable is respiratory variation in impedance, which is used to monitor respiratory rate and volume. As the lungs fill with air during inspiration, impedance increases, and during expiration, impedance decreases. Impedance can be measured during expiration to minimize the effect of breathing on central systemic venous volume. While respiration and CHF both cause variations in impedance, the rates and magnitudes of the impedance variation are different enough to separate out the respiratory variations which have a frequency of about 8 to 60 cycles per minute and congestion changes which take at least several minutes to hours or even days to occur. Also, the magnitude of impedance change is likely to be much greater for congestive changes than for normal respiratory variation. Thus, the system can detect congestive heart failure (CHF) in early stages and alert a patient to prevent disabling and even lethal episodes of CHF. Early treatment can avert progression of the disorder to a dangerous stage.

In an embodiment to monitor wounds such as diabetic related wounds, the conductivity of a region of the patient with a wound or is susceptible to wound formation is monitored by the system. The system determines healing wounds if the impedance and reactance of the wound region increases as the skin region becomes dry. The system detects infected, open, interrupted healing, or draining wounds through lower regional electric impedances. In yet another embodiment, the bioimpedance sensor can be used to determine body fat. In one embodiment, the BI system determines Total Body Water (TBW) which is an estimate of total hydration level, including intracellular and extracellular water; Intracellular Water (ICW) which is an estimate of the water in active tissue and as a percent of a normal range (near 60% of TBW); Extracellular Water (ECW) which is water in tissues and plasma and as a percent of a normal range (near % of TBW); Body Cell Mass (BCM) which is an estimate of total pounds/kg of all active cells; Extracellular Tissue (ECT)/Extracellular Mass (ECM) which is an estimate of the mass of all other non-muscle inactive tissues including ligaments, bone and ECW; Fat Free Mass (FFM)/Lean Body Mass (LBM) which is an estimate of the entire mass that is not fat. It should be available in pounds/kg and may be presented as a percent with a normal range; Fat Mass (FM) which is an estimate of pounds/kg of body fat and percentage body fat; and Phase Angle (PA) which is associated with both nutrition and physical fitness.

The system includes augmenting a wrist-worn jewelry by
wearing the jewelry on a top of the wrist and a digital wearable device secured to the analog watch and on the bottom of the wrist;
reading time on the top of the wrist and turning the wrist to read digital messages generated by the device or messages transmitted from a telephone; and
capturing vital sign or fitness information using the digital wearable device.

The jewelry can be an analog watch and the digital wearable device can be a heart sensor or a heart rate sensor. The system includes perfoming three or more of: analyzing posture, analyzing ambulation, analyzing sleep, analyzing exercise, analyzing user location or position, analyzing walking or running activity, analyzing pedometry or walking pace, analyzing bicycle activity, analyzing calorie consumption, analyzing medicine compliance. More details on techniques (code and processes) to store and analyze user fitness information including one or more of: posture, ambulation, location position, sleep, exercise, calorie, or medicine compliance, are disclosed in co-pending application Ser. No. 14/164,172 filed Jan. 25, 2014, the content of which is incorporated by reference.

In this regard, the FIGS. 1-7 are block diagrams illustrations of methods, systems and program products according to the invention. It will be understood that each block or step of the block diagram and combinations of blocks in the block diagram can be implemented by computer program instructions. These computer program instructions may be loaded onto a computer or other programmable apparatus to produce a machine, such that the instructions which execute on the computer or other programmable apparatus create means for implementing the functions specified in the block diagram, flowchart or control flow block(s) or step(s). These computer program instructions may also be stored in a computer-readable memory that can direct a computer or other programmable apparatus to function in a particular manner, such that the instructions stored in the computer-readable memory produce an article of manufacture including instruction means which implement the function specified in the block diagram, flowchart or control flow block(s) or step(s). The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions specified in the block diagram, flowchart or control flow block(s) or step(s).

Accordingly, blocks or steps of the block diagram, or control flow illustrations support combinations of means for performing the specified functions, combinations of steps for performing the specified functions and program instruction means for performing the specified functions. It will also be understood that each block or step of the block diagram, flowchart or control flow illustrations, and combinations of blocks or steps in the block diagram, flowchart or control flow illustrations, can be implemented by special purpose hardware-based computer systems which perform the specified functions or steps, or combinations of special purpose hardware and computer instructions.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A wearable device, comprising:
a band having a flexible battery integrally formed therein;
a processor powered by the flexible battery;
a cellular transceiver to make a phone call;
a WiFi transceiver and personal area network (PAN) transceiver coupled to the processor;
a microphone to receive user speech and a speech recognizer to process user speech or provide voice control; and
a health application communicating health application data with a remote computer, wherein the health application includes computer readable code to store and analyze user fitness information including one or more of: posture, ambulation, location position, sleep, exercise, calorie, or medicine compliance.

2. The device of claim 1, wherein the device captures heart data using a heart sensor or a heart rate sensor.

3. The device of claim 2, wherein heart data is used to authenticate or identify a user for accessing a computer.

4. The device of claim 1, wherein the digital wearable device comprises a pedometer, a positioning system, or an exercise tracker.

5. The device of claim 1, comprising an energy harvesting device to generate electricity from movement.

6. The device of claim 5, comprising a magnetic pendulum rotor rotatably coupled to a shaft above a planar metal.

7. The device of claim 1, wherein the wrist bands store or generate electrical energy.

8. The device of claim 1, wherein the band comprises a curved flexible display coupled to an power unit to store or generate electrical energy.

9. The device of claim 1, comprising a display circuitry that selectively transitions between being opaque and transparent to enable a user to view a surface under the wearable device.

10. The device of claim 1, comprising a material chamber in the band with one or more chemicals including medicine or adhesive.

11. The device of claim 1, comprising a chamber for collecting tissue material and mixing with a substance for chemical analysis.

12. The device of claim 1, comprising a touch-sensitive flexible display section from display pixel array layer and optional touch sensor layer.

13. The device of claim 1, comprising a touch-sensitive flexible display section with an actuator that deforms the display to provide a desired tactile sensation on the surface of display to a user.

14. The device of claim 1, comprising comprising an actuator that deforms the display to play sound as a speaker.

15. The device of claim 1, comprising an actuator that detects vibrations in the display to capture sound.

16. The device of claim 1, comprising an actuator to temporarily create raised ridges or external features on the surface of the flexible display section.

17. The device of claim 1, comprising apiezoelectric actuator to detect actuation or provide tactile feedback.

18. The device of claim 1, comprising a unit for monitoring the user with a statistical analyzer, a neural network, a back-propagation neural network, a Hidden Markov Model (HMM).

19. The device of claim 18, comprising a unit for authenticating the user user based on ECG, EMG, EEG, or bioimpedance data.

20. The device of claim 18, comprising a unit for detecting a dangerous condition for the user based on ECG, EMG, EEG, or bioimpedance data.
